(19) United States
(12) Patent Application Publication
(10) Pub. No.: US 2010/0324304 A1
(43) Pub. Date: Dec. 23, 2010

(54) IMPROVED RAMIPRIL SYNTHESIS

(76) Inventors: Ben De Lange, Munstergeleen (NL); Hubertus Johnannes A Dielemans, Beek (NL)

Correspondence Address: NIXON & VANDERHYE, PC, 901 NORTH GLEBE ROAD, 11TH FLOOR, ARLINGTON, VA, 22203, US

(21) Appl. No.: 12/742,521

(22) PCT Filed: Nov. 13, 2008
(86) PCT No.: PCT/EP2008/065456

§ 371 Date: Sep. 1, 2010

(30) Foreign Application Priority Data
Nov. 13, 2007 (EP) 07120548.8
Nov. 28, 2007 (EP) 07121771.5

(51) Int. Cl. C07D 209/52

(52) U.S. Cl. 548/452

(57) ABSTRACT

The present invention relates to the preparation of ramipril (formula [1]) from unprotected (2S,3S,6S)-octahydrocyclopenta[b]pyrrole-2-carboxylic acid and to a method for preparing unprotected (2S,3S,6S)-octahydrocyclopenta[b]pyrrole-2-carboxylic acid.

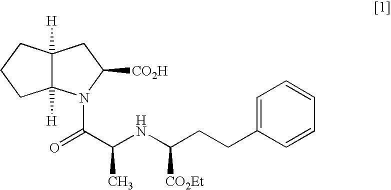

### FIELD OF THE INVENTION

The present invention relates to the preparation of ramipril from unprotected (2S,3S,6S)-octahydrocyclopenta[b]pyrrole-2-carboxylic acid.

### BACKGROUND

Ramipril ([2S,3aS,6aS]-1-[(2S)-2-[[(1S)-1-(ethoxycarbonyl)-3-phenylpropyl]-amino]-1-oxopropyl]octahydrocyclopenta[b]pyrrole-2-carboxylic acid, C₂₃H₃₂N₂O₅, formula [1]) is an angiotensin converting enzyme (ACE) inhibitor.

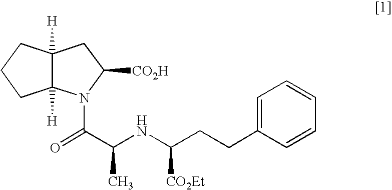

The first synthesis of ramipril was reported in EP 79022 wherein a compound of formula [2] was coupled with a compound of formula [3].

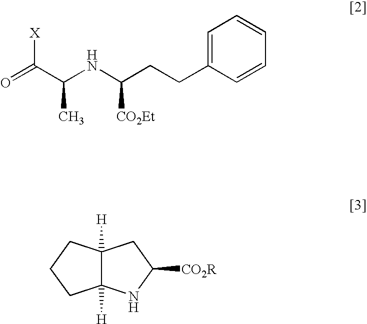

In this synthetic approach X in compound [2] represents OH and coupling was effected through activation, for instance using hydroxybenzotriazole. The R-group in compound [3] represents a carboxyl-protecting group such as benzyl or tert-butyl that is removed after the coupling reaction. The presence of such a carboxyl-protecting group prevents the occurrence of unwanted side reactions. Over the years, improvements in the synthetic protocol have been realized, particularly by activating compound [2] as the halogenide, for example wherein X represents chlorine. Nevertheless, in the demanding arena that the pharmaceutical industry is today, there is an everlasting need for further process improvement.

### DETAILED DESCRIPTION OF THE INVENTION

Despite the fact that (2S,3S,6S)-octahydrocyclopenta[b]pyrrole-2-carboxylic acid ([3], R=H) has been mentioned in EP 79022, the document does not mention or suggest to use this compound for direct coupling with compound [2] to give ramipril. As a matter of fact, protection as a benzyl or tert-butyl ester ([3], R=—CH₂Ph and —C(CH₃)₃, respectively) is even advocated. Only one example exists wherein unprotected (2S,3S,6S)-octahydrocyclopenta[b]pyrrole-2-carboxylic acid ([3], R=H) is coupled to give ramipril. In WO 2007/079871 this is realized using condensation of [3] (R=H) with 2-(4-methyl-2,5-dioxooxazolidin-3-yl)-4-phenylbutyric acid ethyl ester, a compound wherein the secondary amine is protected by means of the use of hazardous phosgene or a derivative made from phosgene in special equipment, contrary to [2] wherein the secondary amine is unprotected.

In the present invention it was found that coupling of the free acid (2S,3S,6S)-octahydrocyclopenta[b]pyrrole-2-carboxylic acid ([3], R=H) with a suitably activated derivative of compound [2] or a salt thereof yielded ramipril in excellent yield and purity. It was thus surprisingly established that compounds [2] or their salts could be coupled directly with compound [3] (R=H) without any secondary amine protection.

Suitably activated derivatives of compound [2] include halogenides, i.e. wherein X is halogen such as bromine, chlorine, fluorine or iodine. Preferred compounds are [2] with X is bromine and [2] with X is chlorine; also mixtures can be used. Said halogenides can be prepared from compound [2] with X is OH using methods that are known to the skilled person. Other suitably activated derivatives of compound [2] are those wherein X is OR₁ with R₁ being C(O)R₂ wherein R₂ represents alkyl, aryl and the like.

(2S,3S,6S)-octahydrocyclopenta[b]pyrrole-2-carboxylic acid ([3], R=H) or suitable salts thereof can be obtained through various approaches. In one approach a corresponding ester is deprotected using methods available to the skilled person. Suitable esters are for instance the benzyl ester ([3], R=—CH₂C₆H₅), the tert-butyl ester ([3], R=—C(CH₃)₃), the 4-nitrobenzyl ester ([3], R=—CH₂C₆H₄NO₂) and similar esters. Deprotection of benzyl and 4-nitrobenzyl esters can be achieved under hydrolytic conditions, for instance using acid or base, or under reductive conditions using any of the well known hydrogenation approaches. Reductive deprotection is favorable as it was observed that formation of unwanted side products usually is less or even completely absent. In one embodiment reductive deprotection of the benzyl ester ([3], R=—CH₂C₆H₆) is carried out using Pd/C and hydrogen in a suitable solvent. Suitable solvents are esters such as ethyl acetate, iso-propyl acetate and the like, halogenated hydrocarbons such as dichloromethane and benzene-derivatives such as toluene and the xylenes.

Alternatively, (2S,3aS,6aS)-octahydrocyclopenta[b]pyrrole-2-carboxylic acid ([3], R=H) or suitable salts thereof can be obtained through an approach in which a suitable precursor, such as for instance a hydantoin like 5-((2-oxocyclopentyl)methyl)imidazolidine-2,4-dione, is enzymatically hydrolyzed to give an optically pure amino acid, like (S)-2-amino-3-(2-oxocyclopentyl)propanoic acid, which then undergoes ring-closure, for instance by hydrogenation, to give (2S,3aS,6aS)-octahydrocyclopenta[b]pyrrole-2-carboxylic acid ([3], R=H) or a suitable salt thereof.

In one embodiment (2S,3S,6S)-octahydrocyclopenta[b]pyrrole-2-carboxylic acid ([3], R=H) is first treated with a base, for instance an alkali alkoxide such as potassium ethoxide, potassium methoxide, sodium ethoxide or sodium methoxide. Preferably the coupling reaction with [2] is carried out also in the presence of an organic base such as, for instance, DBN, DBU, diisopropylethylamine, dimethylethylamine, imidazole, triethylamine and the like. In principle the reaction can be carried out at a wide range of temperatures, however in order to avoid unwanted side reactions it is preferred to start at low temperatures, i.e. ranging from −196° C. to 0° C., preferably ranging from −78° C. to −20° C., more preferably ranging from −50° C. to −20° C.

The product obtainable by the method of the present invention is of pharmaceutically acceptable quality or better. In fact, the method of the present invention avoids the use of protecting groups and thus avoids the use of additional reagents required for removal of protecting groups. Hence, ramipril obtainable by the method of the present invention is free of certain impurities that would normally be present as a result from prior art methods. As a result, the product obtainable by the method of the present invention can be used without additional purification in the preparation of medicaments

The process of the present invention includes the racemic forms as well as the optically pure forms as well as mixtures of the two forms.

### EXAMPLES

### Example 1

### Preparation of (S)-ethyl 2-[(S)-1-(chlorocarbonyl)ethylamino]-4-phenylbutanoate hydrochloride

A 4M HCl solution in 1,4-dioxane (6 mL, 24 mmol) was dosed at 0° C. to a slurry of (S)-2-[(S)-1-(ethoxycarbonyl)-3-phenylpropylamino]propanoic acid (5.58 g, 20 mmol) in dry dichloromethane (30 mL). To the resulting solution, phosphorous pentachloride (5.00 g, 24 mmol) was added, maintaining the temperature at 0° C. The mixture was then allowed to warm up to 20° C. during 30 min and was stirred at that temperature for 16 h. After evaporation of the solvents in vacuo, diethyl ether (50 mL) was added. The suspension was stirred for 30 min at 0° C., filtered, washed with diethyl ether (2×20 mL) and dried under vacuum. The product (S)-ethyl 2-[(S)-1-(chlorocarbonyl)ethylamino]-4-phenyl-butanoate hydrochloride is an off-white solid. It must be stored under nitrogen. Weight 6.23 g (18.6 mmol), 93% yield.

### Example 2

### Biocatalytic preparation of (2S,3aS,6aS)-octahydrocyclopenta[b]pyrrole-2-carboxylic acid

Protocol for Transformation of DKECaroP-hyul Construct into Escherichia coli RV308

Thaw Escherichia coli RV308 aliquots (200 μl, super competent) on ice

Add 15 μl LR reaction mix (see above)

Incubate 30 minutes on ice

Heat shock 1 minute 42° C.

Cool cells 2 minutes on ice

Add 1 ml LB medium (5 g/l NaCl, 5 g/l yeast extract, 10 g/l tryptone)

Incubate 1 hour 37° C.

Plate on LB agar plates supplemented with kanamycine (5 g/l NaCl, 5 g/l yeast extract, 10 g/l tryptone, 15 g/l agar, 50 mg/l kanamycine)

Incubate 24 hours 28° C.

Isolate single colonies

Protocol for Expression of Hyu Genes in Escherichia coli RV308

Single clones from the transformation (see above) were used to inoculate 5 ml of 2×TY media (10 g/l yeast extract, 16 g/l tryptone, 5 g/l NaCl) supplemented with 0.05 g/l kanamycine and 1 mM MnCl₂ or CoCl₂, respectively. The culture was incubated at 28° C. and 150 rpm for 24 hours and then used for inoculation of 100 ml 2×TY media supplemented with 0.05 g/l kanamycine and 1 mM MnCl₂ or CoCl₂, respectively. The cultures were again incubated for 24-28 hours under conditions previously mentioned and subsequently harvested by centrifugation (20 minutes, 5000 rpm, 4° C.). The cell pellet was resuspended in 5 ml Tris-HCl (100 mM, pH 7), centrifuged again (20 minutes, 5000 rpm, 4° C.) and the cells were frozen at −20° C.

### Bioconversion

5-((2-oxocyclopentyl)methyl)imidazolidine-2,4-dione (0.20 g, 1.0 mmol) was suspended in TRIS-HCl buffer (400 mM, 5 mL) at pH 8.5. Then, 0.8 g of wet cell slurry obtained according to ‘Protocol for expression of Hyu genes in Escherichia coli RV308’ was added, followed by 50 μL of a 100 mmol/L MnCl₂ solution and the mixture was incubated overnight at 37° C. The mixture was then centrifuged at room temperature and the supernatant was filtered through a 0.45μ filter. The product amino acid has the S configuration at C2 with >99% ee (the other chiral center C4 is scrambled). The diastereomeric product mixture was further purified by ion exchange column chromatography using acidic DOWEX 50WX2-100 (0.6 meq/mL) resin. Evaporation of the aqueous eluent in vacuo at 80° C. provided the product mixture as an off-brown solid. Weight 137 mg. 80% yield. ¹H NMR: (D₂O, 300 MHz): δ 3.88-3.67 (m, 1H), 2.38-1.48 (m, 9H).

(S)-2-amino-3-(2-oxocyclopentyl)propanoic acid thus obtained (130 mg, 0.76 mmol) was dissolved in water (2 mL) and the pH was adjusted to 9 with 25% aqueous NH₃ solution. Then, 10% Pd/C (5 mg) was added and hydrogenation was performed under 5 bar of hydrogen gas for 16 h. At the end of the reaction, the catalyst was filtered off on a pad of celite under suction and the title product was isolated after evaporation of the water layer in vacuo at 80° C. Weight 118 mg. 100% yield. Diastereomeric excess >95% (determined by chiral HPLC). ¹H NMR: (DMSO-d₆, 300 MHz): δ 10.54 (br s, 1H), 8.71 (br s, 1H), 4.22 (dd, 1H), 3.98 (t, 1H), 2.86-2.76 (m, 1H), 2.49-2.42 (m, 1H), 2.00-1.96 (m, 1H), 1.80-1.40 (m, 6H).

### Example 3

### Preparation of (2S,3aS,6aS)-octahydrocyclopenta[b]pyrrole-2-carboxylic acid hydrochloride by reductive debenzylation of (2S,3aS,6aS)-benzyl octahydrocyclopenta[b]pyrrole-2-carboxylate hydrochloride

(2S,3aS,6aS)-Benzyl octahydrocyclopenta[b]pyrrole-2-carboxylate hydrochloride (20.0 g, 71.0 mmol) was suspended in toluene (150 mL). Then 5% Pd/C (1.00 g, E196 Degussa, 56% water) was added and the reaction mixture was stirred for 18 h at 40° C. under 28 bar H₂ pressure. Upon completion of the reaction, methanol (100 mL) was added to dissolve the insoluble (2S,3aS,6aS)-octahydrocyclopenta[b]pyrrole-2-carboxylic acid hydrochloride (reaction product). The catalyst was filtered off on dicalite and washed with fresh methanol (50 mL). The combined filtrates were evaporated in vacuo (50° C.) and to the residue was added diethyl ether (100 mL). The product was filtered and dried in vacuo at 40° C. Weight 13.0 g (68 mmol), 96% yield. ¹H NMR: (DMSO-d₆, 300 MHz): δ10.4 (s, 1H), 8.7 (s, 1H), 4.3 (m, 1H), 4.0 (m, 1H), 2.8 (m, 1H), 2.45 (m, 1H), 2.0 (m, 1H), 1.4-1.8 (m, 6H).

### Example 4

### Preparation of (2S,3aS,6aS)-octahydrocyclopenta[b]pyrrole-2-carboxylic acid by reductive debenzylation of (2S,3aS,6aS)-benzyl octahydrocyclopenta[b]pyrrole-2-carboxylate

(2S,3aS,6aS)-Benzyl octahydrocyclopenta[b]pyrrole-2-carboxylate hydrochloride (50.0 g, 177 mmol) was dissolved in aqueous NaOH solution (300 mL, 1.5 M, 450 mmol) and the free amine was extracted with CH₂Cl₂ (3×150 mL). The combined organic layers were evaporated in vacuo and the residue (2S,3aS,6aS)-benzyl octahydrocyclo-penta[b]pyrrole-2-carboxylate (43.9 g, 177 mmol) was dissolved in toluene (150 mL). Then 5% Pd/C (1.50 g, E196 Degussa, 56% water) was added. The reaction mixture was stirred for 17 h at 40° C. under 25 bar H₂ pressure. Then methanol (300 mL) was added, the mixture was brought to 60° C. to dissolve the product and the hot mixture was filtered on dicalite. The dicalite cake was re-suspended in methanol (100 mL), stirred at 60° C., filtered and this was repeated once more. The combined methanol filtrates were concentrated in vacuo (45° C.) to a volume of 100 mL. Then diethyl ether (150 mL) was added, the slurry was stirred at 0-5° C. and then filtered to collect the product (2S,3aS,6aS)-octahydrocyclopenta[b]pyrrole-2-carboxylic acid as a solid, which was dried in vacuo. Weight 26.0 g (168 mmol), 95% yield. ¹H NMR: (DMSO-d₆/D₂O, 300 MHz): δ3.9 (m, 1H), 3.7 (m, 1H), 2.7 (m, 1H), 2.35 (m, 1H), 1.9 (m, 1H), 1.3-1.8 (m, 6H).

### Example 5

### Preparation of Ramipril

A 20% (w/w) solution of sodium ethoxide in ethanol (12.1 g, 35.2 mmol) was diluted with dry ethanol (100 mL) and (2S,3S,6S)-octahydrocyclopenta[b]pyrrole-2-carboxylic acid (5.00 g, 32.2 mmol) was added and the solution was cooled to −40° C. Then triethylamine (9.50 g, 93.9 mmol) was added. Subsequently, a slurry of (S)-ethyl 2-[(S)-1-(chlorocar-bonyl)ethylamino]-4-phenylbutanoate hydrochloride (11.3 g, 33.8 mmol, obtained in Example 1) in dry dichloromethane (100 mL) was dosed during 5 min, maintaining the temperature between −50 and −40° C. After 1 h at −40° C., the reaction temperature was allowed to increase to 20° C. and water (250 mL) was added after which the organic solvents were evaporated in vacuo at 45° C. The residual solution was acidified with 2N HCl aq. (25 mL) to pH 4.2. The aqueous phase was then extracted with ethyl acetate (1×250 mL, 3×100 mL). The organic phase was dried with anhydrous magnesium sulfate, filtered and the solvents were evaporated in vacuo at 45° C. The residual oil was dissolved in diethyl ether (100 mL) and seeded with ramipril (10 mg). After crystallization, the precipitate was collected via filtration, washed with diethyl ether (2×20 mL) and dried in vacuo. The product ramipril is a colorless solid. Weight 11.8 g (28.3 mmol). 88% yield. ¹H NMR: (CDCl₃) according to reference. HPLC-purity: 98.0%.

1. A method for the preparation of ramipril comprising contacting (2S,3S,6S)-octahydrocyclopenta[b]pyrrole-2-carboxylic acid or a salt thereof with a compound of general formula [2] or a salt thereof 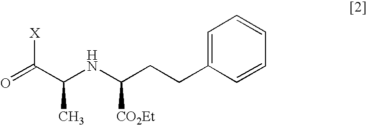
wherein X is halogen or OR₁ with R₁ being C(O)R₂ wherein R₂ represents alkyl or aryl.

2. Method according to claim 1 wherein at least the first 30 minutes are carried out at a temperature ranging from −196° C. to 0° C.

3. Method according to claim 2 wherein said temperature is ranging from −78° C. to −20° C.

4. Method according to claim 3 wherein said temperature is ranging from −50° C. to −20° C.

5. Method according to claim 1 wherein X is bromine or chlorine.

6. Ramipril obtainable by the method of claim 1.

7. Use of a ramipril according to claim 6 in the preparation of a medicament.

8. A method for the preparation of (2S,3S,6S)-octahydrocyclopenta[b]pyrrole-2-carboxylic acid or a salt thereof comprising deprotection of an ester of (2S,3S,6S)-octahydrocyclopenta[b]pyrrole-2-carboxylic acid

9. Method according to claim 8 wherein said ester is (2S,3aS,6aS)-benzyl octahydrocyclopenta[b]pyrrole-2-carboxylate or a salt thereof and wherein said deprotection is carried out by hydrogenation.

10. Use of (2S,3S,6S)-octahydrocyclopenta[b]pyrrole-2-carboxylic acid or a salt thereof and a compound of general formula [2] or a salt thereof according to claim 1 in the preparation of ramipril.
